(19) United States
(12) Patent Application Publication
(10) Pub. No.: US 2019/0224038 A1
(43) Pub. Date: Jul. 25, 2019

(54) Reusable penile attachment

(71) Applicant: Gerald Mayrand, St-Hubert (CA)

(72) Inventor: Gerald Mayrand, St-Hubert (CA)

(21) Appl. No.: 16/254,594

(22) Filed: Jan. 23, 2019

(30) Foreign Application Priority Data
Jan. 24, 2018 (GB) 1801184.1

(51) Int. Cl. A61F 5/453; A61F 5/44

(52) U.S. Cl. CPC A61F 5/453; A61F 5/4408

(57) ABSTRACT

An improved and reusable penile attachment is provided. The penile attachment is in the form of a cup of approximately 1″⅜ depth with a wider opening for inserting the penis of approximately 1″¼ on one end and a narrow opening of approximately ⅜″ with an extension for attaching the tube on the other end. The invention is cupped onto the glans or head of the penis using a lubricant. The narrow end with extension is connected via a tube to a collection bag which is strapped onto the user's leg using two straps. Similarly, the invention is pulled off the penis glans when detaching and can be washed and reused several times.

### CROSS-REFERENCE TO RELATED APPLICATIONS

The present application claims priority to United Kingdom Patent Application serial number GB1801184.1, filed on Jan. 24, 2018 entitled “An improved and reusable penile attachment”, the disclosure of which is hereby incorporated in its entirety at least by reference.

### BACKGROUND OF THE INVENTION

### 1. Field of the Invention

The present invention relates generally to a penile sheath or attachment for incontinence but more particularly to an improved and reusable penile attachment.

### 2. Description of Related Art

Urinary incontinence (UI), also known as involuntary urination, is any uncontrolled leakage of urine. Incontinence among men is a growing problem worldwide and has been identified as an important issue in geriatric health care.

In order to improve the quality of life in men suffering from incontinence, there exist several solutions ranging from penile clamps for simple dripping to sheaths connected to a bag for more serious cases of incontinence. In cases where a sheath or penile attachment is connected via a tube to a bag, the penile attachment is to be changed every 24 hours to a maximum of 48 hours in some devices. The methods of affixing the attachment to the penis include, glue, straps, clamps etc., which are all uncomfortable. Additionally, it can take time fitting and removing the device and requires the pubic area to be shaved for some attachments. Furthermore, these attachments are prone to slipping off and unsuitable for sporting and other activities which require movement.

There hence exists a need for an improved penile attachment which is quick to fix, suitable for a variety of movements and reusable, thereby enhancing the quality of life for men of all ages suffering from incontinence.

### BRIEF SUMMARY OF THE INVENTION

In one aspect of the invention an improved penile attachment for urinary incontinence is provided, comprising a cup having a depth comprising an opening having a first width and an exit having a second width, wherein the first width is greater than the second width; a tube having a first end and a second end, wherein the first end is attached to the exit; a collection bag having attachment straps, wherein the collection bag is attached to the second end of the tube; and, wherein the opening of the cup is configured to fit onto a glans of a penis, and the collection bag is configured to collect urine leakage.

In one embodiment, the attachment straps are configured to attach to a leg. In one embodiment, the improved penile attachment may be washed and reused. In one embodiment, the depth is approximately 1⅜″, the first width is approximately 1¼″, and the second with is approximately ⅜″.

In another aspect of the invention, a method to collect urinary leakage of a user is provided, comprising steps (a) providing a cup having a wide opening and a narrow exit attached to a collection bag via a tube; (b) affixing the wide opening to a glans of a penis of the user; and, (c) attaching the collection bag to a leg of the user.

In one embodiment, in step (b), the affixing is without the use of glue. In one embodiment, in step (c), the attaching is via attachment straps.

### BRIEF DESCRIPTION OF THE SEVERAL VIEWS OF THE DRAWINGS

Other features and advantages of the present invention will become apparent when the following detailed description is read in conjunction with the accompanying drawings, in which:

FIG. 1 is a front view of an improved and reusable penile attachment showing the collection bag and tubing according to an embodiment of the present invention.

FIGS. 2A-B illustrate the improved and reusable penile attachment in use according to an embodiment of the present invention.

FIG. 3A is a cutaway view of a first penile attachment taken along A-A of FIG. 3B.

FIG. 3B is a side view of the first penile attachment according to an embodiment of the present invention.

FIG. 4A is a cutaway view of a second penile attachment taken along A-A of FIG. 4B.

FIG. 4B is a side view of the second penile attachment according to an embodiment of the present invention.

### DETAILED DESCRIPTION OF THE PREFERRED EMBODIMENT

The following description is provided to enable any person skilled in the art to make and use the invention and sets forth the best modes contemplated by the inventor of carrying out their invention. Various modifications, however, will remain readily apparent to those skilled in the art, since the general principles of the present invention have been defined herein to specifically provide an improved and reusable penile attachment.

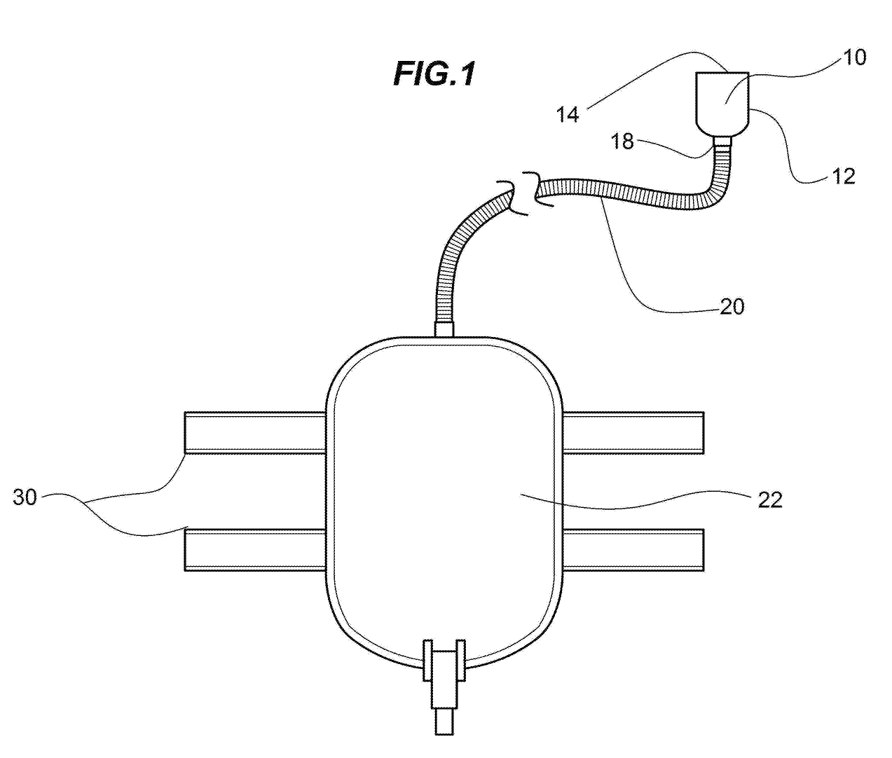

Referring now to FIG. 1, an improved and reusable penile attachment 10 is illustrated. The penile attachment comprises a cup 12 having a wide opening 14 and a narrow exit 18. In one embodiment, the narrow exit is coupled and affixed to a tube 20 which connects to a collection bag 22. The collection bag includes attachment straps 30 for attaching the collection bag to the user. This will be described in greater detail below.

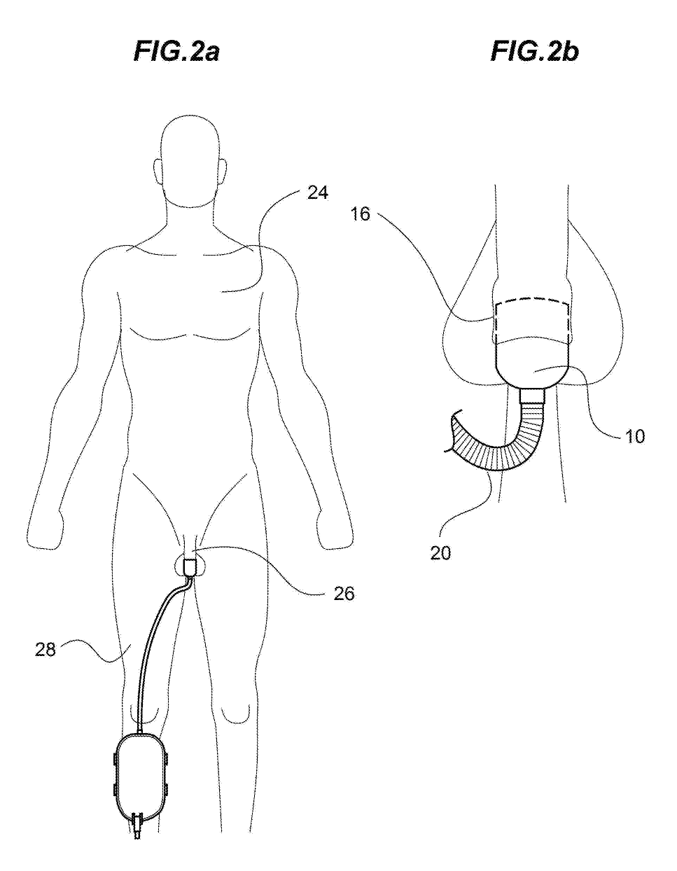

Referring now to FIGS. 2A-B, the improved and reusable penile attachment is in use. During use, opening 14 is configured to fit onto a glans 16 of a penis 26 of a user 24. Collection bag 22 is then attached to a suitable portion of the user's leg 28. It should be understood, that the location of collection bag may vary. In one embodiment, the straps have hook and loop fasteners, or other suitable attachment means known in the art to affix the straps to each other. Similarly, the reverse is done when the user detaches the device. In one embodiment, a lubricant (not shown) is used when affixing the cup on the glans of the penis. Advantageously, the penile attachment remains affixed until it is physically pulled off or detached without the use of glue. After detachment, the penile attachment may be washed and reused.

In one embodiment, the depth of the cup is approximately 1⅜″, the wide opening is approximately 1¼″ and the narrow exit is approximately ⅜″. Although, the preferred dimensions are disclosed, it should be understood the values may vary without departing from the spirit and scope of the invention.

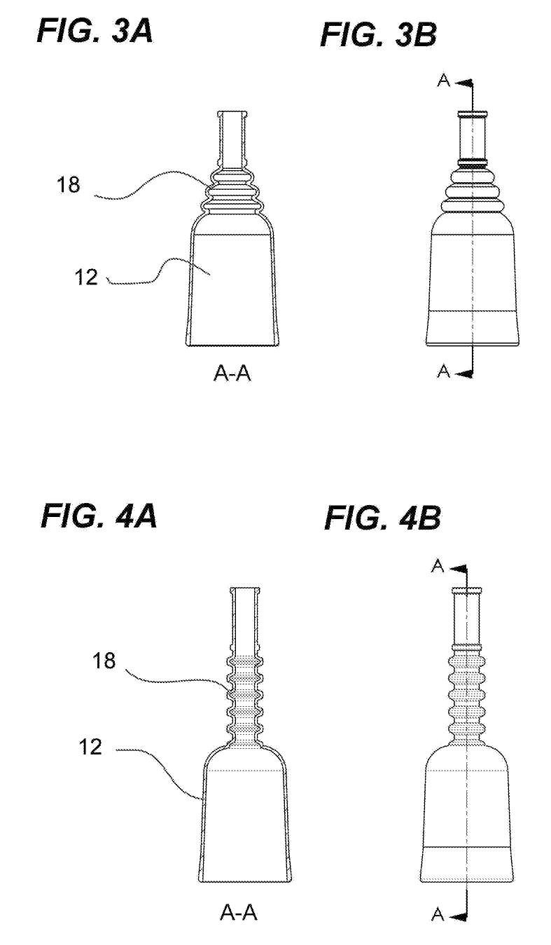

Referring now to FIGS. 3A-B and 4A-B, show a first and second penile attachment design respectably. As illustrated, both designs have similar cup 12 designs however they each have different narrow exit 18 designs. The second penile attachment has a longer and narrower accordion style exit having generally the same width until meeting with the tube. The first penile attachment is shorter and has a descending width until meeting with the tube. It should be understood, that the two designs are just exemplary embodiments, and other designs are possible.

Although the invention has been described in considerable detail in language specific to structural features and or method acts, it is to be understood that the invention defined in the appended claims is not necessarily limited to the specific features or acts described. Rather, the specific features and acts are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated, and can be made without departing from the spirit and scope of the invention.

It should further be noted that throughout the entire disclosure, the labels such as left, right, front, back, top, bottom, forward, reverse, clockwise, counter clockwise, up, down, or other similar terms such as upper, lower, aft, fore, vertical, horizontal, oblique, proximal, distal, parallel, perpendicular, transverse, longitudinal, etc. have been used for convenience purposes only and are not intended to imply any particular fixed direction or orientation. Instead, they are used to reflect relative locations and/or directions/orientations between various portions of an object.

In addition, reference to “first,” “second,” “third,” and etc. members throughout the disclosure (and in particular, claims) are not used to show a serial or numerical limitation but instead are used to distinguish or identify the various members of the group.

What is claimed is:

1. An improved penile attachment for urinary incontinence comprising:
a cup having a depth comprising an opening having a first width and an exit having a second width, wherein the first width is greater than the second width;
a tube having a first end and a second end, wherein the first end is attached to the exit;
a collection bag having attachment straps, wherein the collection bag is attached to the second end of the tube; and,
wherein the opening of the cup is configured to fit onto a glans of a penis, and the collection bag is configured to collect urine leakage.

2. The improved penile attachment of claim 1, wherein the attachment straps are configured to attach to a leg.

3. The improved penile attachment of claim 1, wherein the improved penile attachment may be washed and reused.

4. The improved penile attachment of claim 1, wherein the depth is approximately 1⅜″, the first width is approximately 1¼″, and the second with is approximately ⅜″.

5. A method to collect urinary leakage of a user comprising steps:
(a) providing a cup having a wide opening and a narrow exit attached to a collection bag via a tube;
(b) affixing the wide opening to a glans of a penis of the user; and,
(c) attaching the collection bag to a leg of the user.

6. The method of claim 5, wherein in step (b), the affixing is without the use of glue.

7. The method of claim 5, wherein in step (c), the attaching is via attachment straps.
